(19) United States
(12) Patent Application Publication
(10) Pub. No.: US 2007/0254320 A1
(43) Pub. Date: Nov. 1, 2007

(54) Method and Kit for Detecting Listeria Spp.

(76) Inventor: Alan Olstein, Mendota Heights, MN (US)

Correspondence Address: INTELLECTUAL PROPERTY DEPARTMENT;DEWITT ROSS & STEVENS, S.C., EXCELSIOR FINANCIAL CENTRE, 8000 EXCELSIOR DRIVE, SUITE 401, MADISON, WI, 53717-1914, US

(21) Appl. No.: 10/576,843

(22) PCT Filed: Oct. 20, 2004
(86) PCT No.: PCT/US04/34633

§ 371 Date: Mar. 8, 2007

Related U.S. Application Data
(60) Provisional application No. 60/512,730, filed on Oct. 20, 2003.

(51) Int. Cl. G01N 33/00

(52) U.S. Cl. 435/7.32

(57) ABSTRACT

The subject invention is directed to a method and a kit for detecting Listeria spp. in food samples, biological sample (e.g., blood, saliva, tissue samples, cells samples, etc.), and any other sample suspected of containing Listeria.

### FIELD OF THE INVENTION

The subject invention is directed to a method and a corresponding kit for detecting Listeria spp. in food samples, biological samples (e.g., blood, saliva, tissue samples, cells samples, etc.), and any other sample suspected of containing Listeria. The method yields accurate results very quickly (about 1 hour) and the method is highly sensitive (e.g., a detection limit of about 10 to 50 colony forming units) as compared to prior art assays.

### BACKGROUND OF THE INVENTION

Conventional tests to detect bacterial pathogens in food typically take about 24 to 72 hours to complete. The time lag between the sampling of the product and the acquisition of the test results presents difficult logistical and public health problems.

For example, in the standard FDA procedure for detection of Listeria in food products, a 25 g or 25 ml aliquot is mixed with 225 ml of enrichment broth. The broth mixture is incubated for 2 days. At the end of Day 1 and Day 2, a sample of the broth is streaked onto petri plates containing a medium selective for Listeria growth. The plates are then incubated for an additional 2 days (see “Bacteriological Analytical Manual,” 7th Ed., 1992; Chapter 10). Growth of Listeria colonies on the plates confirms the presence of Listeria in the original food sample. The identification, however, is subjective, and colonies that do appear are often further tested to confirm their identity. At minimum, therefore, the FDA-mandated procedure requires 4 days to confirm Listeria-negative samples.

On one hand, it is technically possible to impound food products until they have been adequately tested for bacterial contamination. But this is not logistically feasible because many agricultural products spoil quickly and thus must appear on the supermarket shelves without delay. Refrigerated warehouses are not necessarily equipped to hold a large volume of impounded product while new product continues to arrive at the warehouse loading docks. Moreover, a host of state and federal regulations put limits on how long certain products, such as milk, can be offered for sale before they must be discarded (regardless of whether the product is spoiled or not). Thus, impounding the product until testing is complete is not an ideal solution.

On the other hand, the products can be shipped to supermarkets for sale and recalled only if the tests come back positive for bacterial contamination. However, in today's mass market economy, food products are transported vast distances prior to their ultimate sale to consumers. For example, it is possible in the northern hemisphere to get out-of-season table grapes shipped all the way from southern hemisphere countries such as Chile and Argentina. If some of these products should prove to be tainted, tracking the ultimate destination of the tainted products presents a daunting public health quandary. Which products must be discarded, which products are untainted?

In the case of Listeria specifically, the problem is especially acute because infection by Listeria yields a death rate of roughly 40% in human populations at large. The mortality rate among infected newborns, pregnant women, the elderly and immuno-compromised individuals is much higher. Listeria is known to induce spontaneous abortion.

There are a number of U.S. Patents that describe different technical means to attempt to address the problem of detecting Listeria. For example, Butman et al., U.S. Pat. No. 4,950,489, describe an ELISA-based method for detecting Listeria. Green et al., U.S. Pat. No. 5,139,993, describe a detection method wherein selective antibodies are used to capture the peptidoglycan and teichoic acid components of the Listeria bacterial cell wall. Stackebrandt et al, U.S. Pat. No. 5,089,386, and Blais, U.S. Pat. No. 5,827,661, employ nucleic acid hybridization techniques. Metabolic approaches, such as those described in Bochner, U.S. Pat. No. 5,134,063, and Facon et al., U.S. Pat. No. 6,228,606, required prolonged growth periods (e.g., at least 24 hours) to achieve a threshold mass of viable cells for the assays to yield useful results. As a general proposition, most prior art analytical methods for detecting Listeria spp. that require a culture period have a detection limit of roughly 10⁵ to 10⁷ colony-forming units.

All of the methods cited above suffer from a lack of sensitivity. They are also time-consuming, generally taking at least 24 hours to complete. The prior art methods described above cannot be used to analyze directly an environmental or food sample isolate and obtain a useful result in less than twenty-four hours. Thus there remains a long-felt and unmet need for a method to detect Listeria spp. that is fast, sensitive, accurate, and precise. The method disclosed herein is all of these.

### SUMMARY OF THE INVENTION

As described in full below, a first embodiment of the invention is directed to a method for detecting Listeria spp. in a sample. The method comprises providing an inert surface having adhered thereto anti-Listeria antibodies capable of capturing Listeria spp. cells and contacting the surface with a sample suspected of containing Listeria spp., wherein Listeria spp. cells present in the sample adhere to the anti-Listeria antibodies on the surface. The surface is then contacted with a substrate for beta-glucosidase that produces luminescence when hydrolyzed. In this fashion, beta-glucosidase produced by the Listeria spp. cells adhered to the anti-Listeria antibodies catalyzes hydrolysis of the substrate, thereby generating luminscence. The surface is also contacted with an enhancer molecule to enhance and stabilize the luminscence. The luminescence so generated is then detected, wherein the luminescence is indicative of the presence of the Listeria spp. cells in the sample.

The method may also include the optional step of aging the substrate at room temperature in the presence of proteins for a period of at least 12 hours, and preferably 24 hours or more. (See Example 4 below.)

The method may also include separating the surface from the sample after the surface has been contacted with the sample to be tested.

A second embodiment of the invention is directed to a kit for detecting Listeria spp. in a sample. The kit comprises an inert surface having adhered thereto anti-Listeria antibodies capable of capturing Listeria spp. cells; a substrate for beta-glucosidase that produces luminescence when hydrolyzed, wherein the substrate is disposed in a first container; an enhancer molecule disposed in a second container, and instructions for use of the kit.

### BRIEF DESCRIPTION OF THE DRAWING FIGURES

FIG. 1 is a graph depicting the performance of different types of particles for recovering the target bacteria. Silica-dextran (SiDe)-coated particles yielded the highest recovery of target bacteria; see Example 1.

FIG. 2 is a graph depicting the chemiluminescent titration curve for the cell dilutions of pure Listeria cultures described in Example 2.

FIG. 3 is a graph depicting the effect of adding chymotrypsin to the chemiluminescent reaction.

FIG. 4 is a graph depicting the effect of adding bovine serum albumin (BSA) to the chemiluminescent reaction.

### DETAILED DESCRIPTION OF THE INVENTION

The invention disclosed herein is a method for detecting Listeria spp. The method yields definitive results, generally within about one (1) hour, with a detection limit in the range of from about 10 to about 50-colony forming units. As noted above, most prior art analytical methods for detecting Listeria spp. have a detection minimum of 10⁵ to 10⁷ colony-forming units. This fact is the principal reason why cultural enrichment in selective media is required in the prior art methods for detecting the pathogen.

Additionally, most users want to distinguish the number and type of viable organisms (e.g. pathogens vs. non-pathogens) from non-viable organisms. As describe in greater detail below, the present invention ensures that the pathogen detected is also a viable organism. Additionally, the present invention brings to bear several different means of selection to detect and remove contaminants from the sample, thereby increasing the likelihood that the presumptive positive test in the initial screening test is a true positive.

The present invention thus provides a method to obtain a presumptive positive result for detecting viable Listeria spp. within one (1) hour, and a confirmed result within twenty-four hours. The one-hour screening test comprises identifying viable Listeria via an exquisitely sensitive assay for the Listeria-associated enzyme, β-glucosidase.

Recently, a new family of 1,2 dioxetane derivatives which act as chemiluminescent substrates for enzymatic reactions involving β-glucosidase have become commercially available. These derivatives exhibit substantially greater signal enhancement under assay conditions to permit single molecule detection. Brij et al. have published data demonstrating the sensitivity of this system for the chemiluminescent detection of single molecules of alkaline phosphatase. See Brij et al., PCT/US99/20590 and Brij et al., U.S. Published Patent Application 2002/0013250. In short, any chemiluminescent, beta-glucosidase cleavable, 1,2-dioxetane compound that can be used in practicing the present invention.

Note, however, that the ability to hydrolyze several types of aromatic glucosides is not unique to Listeria spp. Many micro-organisms found in the environment can hydrolyze these compounds to obtain metabolic energy from the resulting glucose product. To increase the selection for Listeria in the present invention, the present method preferably uses immuno-magnetic particles prepared from paramagnetic particles coated with a silica-dextran co-polymer. These particles can be obtained commercially from Micromod Partikeltechnologie, GmbH (Rostock, Germany), and are marketed under the “NANOMAG”® and “MICROMER”® registered trademarks. These coated particles exhibit substantially greater capture efficiency toward the target microorganisms as compared commercially available paramagnetic particles coated with plastics such as polystyrene and polystyrene-maleic anhydride co-polymers. The present inventors have found that coupled procedures (use of improved substrates in more selective capture beads) permits detection of as few as about 10 Listeria colony forming units per sample in as little as about 90 minutes, and preferably in as little as about 60 minutes.

While coated particles are preferred, any suitable inert surface and surface geometry can be utilized. Thus, while magnetic particles are preferred, coated beads, rods, pins, disks, plates, microtiter plates, wells, etc., magnetic or non-magnetic, porous or non-porous, can be used with equal success in the present method.

The method also uses an enhancer molecule or molecules to stabilize the resulting chemilluminscense. The enhancer is preferably a synthetic oligomeric or polymeric, water-soluble poly(vinylaryl quaternary ammonium salts), such as poly(vinylbenzyl quaternary ammonium salts) of Formula I:

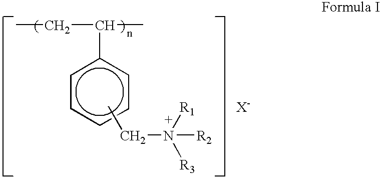

In Formula I each of R₁, R₂ and R₃ can be a straight or branched chain unsubstituted alkyl group having from 1 to 20 carbon atoms, inclusive, e.g., methyl, ethyl, n-butyl, t-butyl, cetyl, or the like; a straight or branched chain alkyl group having from 1 to 20 carbon atoms, inclusive, substituted with one or more hydroxy, alkoxy, e.g., methoxy, ethoxy, benzyloxy or polyoxethylethoxy, aryloxy, e.g., phenoxy, amino or substituted amino, e.g., methylamino, amido, e.g., acetamido or cholesteryloxycarbonylamido, or fluoroalkane or fluoroaryl, e.g., heptafluorobutyl, groups, an unsubstituted monocycloalkyl group having from 3 to 12 ring carbon atoms, inclusive, e.g., cyclohexyl or cyclooctyl, a substituted monocycloalkyl group having from 3 to 12 ring carbon atoms, inclusive, substituted with one or more alkyl, alkoxy or fused benzo groups, e.g., methoxycyclohexyl or 1,2,3,4-tetrahydronaphthyl, a polycycloalkyl group having 2 or more fused rings, each having from 5 to 12 carbon atoms, inclusive, unsubstituted or substituted with one or more alkyl, alkoxy or aryl groups, e.g., 1-adamantyl or 3-phenyl-1-adamantyl, an aryl, alkaryl or aralkyl group having at least one ring and from 6 to 20 carbon atoms in toto, unsubstituted or substituted with one or more alkyl, aryl, or fluoroalkane or fluoroaryl groups, e.g., phenyl, naphthyl, pentafluorophenyl, ethylphenyl, benzyl, hydroxybenzyl, phenylbenzyl or dehydroabietyl; at least two of R₁, R₂ and R₃, together with the quaternary nitrogen atom to which they are bonded, can form a saturated or unsaturated, unsubstituted or substituted nitrogen-containing, nitrogen and oxygen containing or nitrogen and sulfur-containing ring having from 3 to 5 carbon atoms, inclusive, and 1 to 3 heteroatoms, inclusive, and which may be benzoanylated, e.g., 1 pyridyl,1-(3-alkyl or aralkyl)imidazolium, morpholino, piperidino or acylpiperidino, benzoxazole, benzthiazole or benzamidazole.

The symbol X−represents a counterion which can include, alone or in combination, moieties such as halide, i.e., fluoride, chloride, bromide or iodide, sulfate, alkylsulfonate, e.g., methylsulfonate, arylsulfonate, e.g., p-toluenesulfonate, substituted arylsulfonate, e.g., anilinonaphthylenesulfonate (various isomers), lucifer yellow CH and diphenylanthracenesulfonate, perchlorate, alkanoate, e.g., acetate, arylcarboxylate, e.g., fluorescein or fluorescein derivatives, benzoheterocyclicarylcarboxylate, e.g., 7-diethylamino-4-cyanocoumarin-3 carboxylate, phosphate, or substituted monoaryloxyphosphate, e.g., a 3-(2′spiroadamantane)-4-methoxy-(3″-phosphoryloxy)phenyl-1,2-dioxetane dianion or other dianions.

The symbol n represents a number such that the molecular weight of such poly(vinylbenzyl quaternary ammonium salts) will range from about 800 to about 200,000, and preferably from about 20,000 to about 70,000, as determined by intrinsic viscosity or low-angle laser light scattering (LALLS) techniques.

Illustrative of such water soluble poly(vinylbenzyl quaternary ammonium salts) are TMQ, BDMQ, and the like.

These vinylbenzyl quaternary ammonium salt polymers can be prepared by free radical polymerization of the appropriate precursor monomers or by exhaustive alkylation of the corresponding tertiary amines with polyvinylbenzyl chloride. This same approach can be taken using other polymeric alkylating agents such as chloromethylated polyphenylene oxide or polyepichlorohydrin. The same polymeric alkylating agents can be used as initiators of oxazoline ring-opening polymerization, which, after hydrolysis, yields polyethyleneimine graft copolymers. Such copolymers can then be quaternized, preferably with aralkyl groups, to give the final polymeric enhancer substance.

Water soluble acetals of a polyvinylalcohol and a formylbenzyl quaternary ammonium salts, as shown in Formula II, can also be used as enhancer substances in practicing this invention 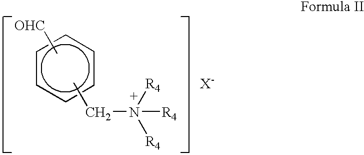
wherein each R₄ is the same or a different aliphatic substituent and X₁ is an anion, as disclosed and claimed in Bronstein-Bonte et al U.S. Pat. No. 4,124,388. And, the individual vinylbenzyl quaternary ammonium salt monomers used to prepare the poly(vinylbenzyl quaternary ammonium salts) of Formula I above can also be copolymerized with other vinylbenzyl quaternary ammonium salt monomers whose polymers are depicted in Formula I, or with other ethylenically unsaturated monomers having no quaternary ammonium functionality, to give polymers such as those disclosed and claimed in Land et al U.S. Pat. No. 4,322,489; Bronstein-Bonte et al U.S. Pat. No. 4,340,522, Land et al U.S. Pat. No.4,424,326; Bronstein-Bonte et al U.S. Pat. No. 4,503,138 and Bronstein-Bonte U.S. Pat. No. 4,563,411, all of which polymers can also be used as enhancer substances in practicing this invention.

Preferably these quaternized polymers will have molecular weights within the ranges given above for the poly(vinylbenzyl quaternary ammonium salts) of Formula I.

Other water soluble oligomeric, homopolymeric and copolymeric materials can be used as enhancer substances in addition to or instead of the foregoing polymers, including:

poly-N-vinyl oxazolidinones; polyvinyl carbamates (e.g., polyvinyl propylene carbamate); polyhydroxyacrylates and methacrylates [e.g., poly(.beta.-hydroxyethyl)methacrylate and polyethyleneglycol monomethacrylates]; amine-containing oligomers (e.g., Jeffamines) quaternized with alkylating or aralkylating agents; synthetic polypeptides (e.g., polylysine co phenylalanine); polyvinylalkylethers (e.g., polyvinyl methyl ether); polyacids and salts thereof [e.g., polyaorylic acids, polymethacrylic acids, polyvinylbenzoic acid, polyethylenesulfonic acid, polyacrylamidomethylpropanesulfonic acid, polymaleic acid and poly(N-vinyl succinamidic acid)]; polyacrylamides and polymethacrylamides derived from ammonia or cyclic and acyclic primary or secondary amines; polyvinyl alcohol and polyvinyl alcohol copolymers with vinyl acetate, ethylene and the like; poly 2-,3- or 4-vinylpyridinium salts where the heterocyclic nitrogen atom is bonded to a group as defined for R₁, R₂, and R in Formula I above; polyvinylalkylpyrrolidinones (e.g., polyvinylmethyl-pyrrolidinones); polyvinylalkyloxazolidones (e.g., polyvinylmethyloxazolidones); branched polyethyleneimines, acylated branched polyethyleneimines, or acylated branched polyethyleneimines further quaternized with alkyl or aralkyl groups; poly N-vinylamines derived from ammonia or cyclic and acyclic primary or secondary amines, and salts thereof; polyvinylpiperidine; polyacryloyl, polymethacryloyl or 4-vinylbenzoyl aminimides where the three substituents on the positively charged nitrogen atom may be any of the R₁, R₂ and R₃ groups defined in Formula I above.

Here too, these oligomeric or polymeric enhancer substances preferably will have molecular weights within the ranges given above for the poly(vinylbenzyl quaternary ammonium salts) of Formula I.

Water soluble monomeric quaternary soaps whose nitrogen atom has at least one benzyl substituent and in which the remaining nitrogen substituents correspond to the definitions given for R₁, R₂, R₃ and X in Formula I, hereinabove, e.g., cetyldimethylbenzylammonium chloride or cetyldibenzylmethyl ammonium bromide, can also be used as enhancer substances when practicing this invention.

The amount of enhancer substance or mixture of enhancer substances employed when practicing this invention can vary within wide limits depending on the particular enhancer substance(s) chosen, the amount and type of chemiluminescent compound(s) present, etc. In general, however, amounts of enhancer substance ranging from about 0.01% to about 25%, and preferably from about 0.1% to about 5%, based on the weight of enhancer divided by the weight of aqueous medium, will be employed.

### EXAMPLES

Particles were obtained from Micromod Partikeltechnologie, as noted earlier. The “NANOMAG”® brand silica particles exhibited the best properties with respect to Listeria capture efficiency (see Example 1). These particles were compared to particles coated with only dextran, with polystyrene-maleic anhydride co-polymer, and with a polylactide polymer. The surface carboxyl groups of the particles were activated with a water-soluble carbodiimide and coupled to protein A (obtained commercially from US Biological, Swampscott, Mass.). The protein A-containing particles were purified magnetically and re-suspended in HEPES buffer, pH 7.0, containing 50 μg/mL anti-Listeria IgG obtained commercially from Toxin Technologies (Sarasota, Fla.). After brief coupling, the particles were magnetically purified and re-suspended in the same volume of phosphate buffer, pH 6.5 containing 0.01% sodium azide and 0.05% Tween-20.

The substrates for β-glucosidase were obtained from Michigan Diagnostics (Troy, Mich.). An assay of Listeria-derived glucosidase was conducted as described in Example 2.

### Example 1

### Capture Efficiency of Immuno-Magnetic Particles

Magnetic particles obtained from Micromod were modified as described previously and coupled with anti-Listeria IgG. Listeria monosytogenes, a laboratory isolate, was grown in trypticase soy broth at 32° C. for eighteen hours in the presence of an isolated environmental contaminant. Cells were serially diluted in 0.1% peptone water, and an aliquot was plated on PALCAM agar plates for counting. PALCAM agar is selective differential medium for the isolation of Listeria monocytogenes from food, clinical and environmental specimens. Selectivity is achieved by the combination of antibiotic supplements and microaerobic incubation. The double indicator system of aesculin hydrolysis and mannitol fermentation aids differentiation of Listeria spp. from enterococci and staphylococci which can be confused with Listeria spp. on other types of culture media. The name PALCAM derives from the various reagents added to the agar: Polymyxin, Acriflavine, Lithium chloride, Ceftazidine, Aesculin, and Mannitol. It is available commercially from several suppliers.

Estimated cell numbers of Listeria and contaminant added to each 15 μL of particles were 1,000 and 10,000,000 cells, respectively, n=2. Fifteen microliters was added to 0.5 mL of the serially-diluted cell suspensions and agitated for thirty minutes at room temperature. The particle-bacteria complexes were collected magnetically and washed twice with 1.5 mL HEPES buffer. The particles were re-suspended in 1.0 mL of HEPES buffer and plated on PALCAM agar and incubated a minimum of eighteen hours for determination of bacterial capture.

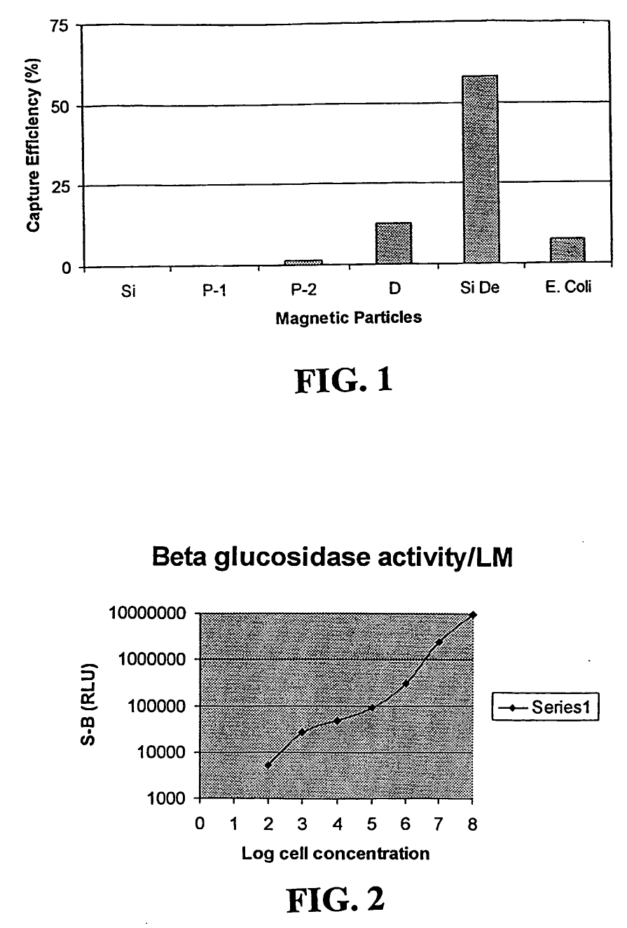

FIG. 1 depicts the data comparing the different types of particle base-material coating. Clearly, the silica-dextran (SiDe) coated particles yielded the highest recovery of target bacteria. In contrast, the other particle materials tested, unmodified dextran (D), unmodified silica (Si) and two plastic materials (P-1, P-2) exhibited far lower capture efficiency. Thus, the SiDe particle is preferred.

### Example 2

### Sensitivity Limit for the Listeria β-Glucosidase Enzyme

Listeria monocytogenes cells of Example 1 were serially diluted as described in Example 1. The G-8-β-glucoside, {(4-(2-phenoxyethoxy)-4-(3-phosphoryloxy-4-chlorophenyl)} spiro {1,2-dioxetane-3,13′-tricyclo{7.3.1.0^{2,7}}tridec-2,7-ene}, disodium salt, was purchased from Michigan Diagnositcs. Eight (8) milligrams of the glucoside was dissolved in 0.5 mL of dimethyl sulfoxide and further diluted into 100 mL of HEPES buffer, pH 7.0. Fifty (50) microliters of each of the cell dilutions was mixed with 0.1 mL of the substrate solution and incubated for sixty minutes at 32° C.

The samples were then removed from the incubator and 0.1 mL of a 1 mg/miL solution of the “enhancer” polymer dissolved in 1M tris-HCl buffer, pH 9.6, was added. The enhancer polymer is a co-polymer of styrene and a polymerizable quaternary ammonium monomer. The preferred enhancer is a poly(vinylbenzyl) ammonium polymer having an weight average molecular weight (M_{w}) of from about 50,000 to 70,000 Da. The polymer is freely water soluble and contains no fluorescent or luminescent moieties within its structure. A host of suitable enhancer polymers are described in U.S. Pat. No. 5,145,772, issued Sep. 8, 1992, to Voyta et al. The same is incorporated herein by reference.

Suitable enhancer polymers can also be obtained commercially from sources such as Applied BioSystems, Inc. (marketers of Nitro-Block II-, Sapphire II, and Emerald II-brand enhancers) (Foster City, Calif.). Suitable enhancers include, without limitation, those falling within the scope of Formulas I and II, hereinabove, e.g., oligomeric and polymeric quaternary ammonium salts, such as poly(vinylaryl quaternary ammonium salts), including poly(vinylbenzyltrimethylammonium chloride) and poly(vinylbenzyl(benzyldimethylammonium chloride.

The sample was then placed in a Berthold Junior 9506 luminometer.

FIG. 2 depicts the chemiluminescent titration curve for the cell dilutions of pure Listeria cultures. The data presented in this titration indicate a minimum detectable signal arising from 50 colony forming unites of Listeria monocytogenes cells. This example demonstrates the utility of this enzyme assay as a one-hour test to detect as few as 50 viable L. monocytogenes bacteria.

### Example 3

### Listeria Analysis of Environmental Samples From a Poultry Plant

Aliquots of environmental samples (500 μL each) obtained from a local poultry processing plant were treated with 30 μL of the silica-dextran particles of Example 1 for sixty minutes at room temperature. The samples were washed twice with HEPES buffer and re-suspended in 1.0 mL of the same buffer. Fifty (50) μL of the sample was placed in a glass tube and 0.1 mL of the β-glucosidase substrate of Example 2 was added. This mixture was then incubated at 30° C. sixty minutes. The samples were brought to room temperature and 0.1 mL of the enhancer polymer added prior to reading the luminescence as described in Example 2.

Table 1 depicts the data obtained from this analysis. Comparison of the signal background ratios yielded three positive samples, and three presumptive positives (because of the small increase over background signal). These samples were found to subsequently have two confirmed positives for Listeria spp. by PALCAM plating of the enriched samples. One of the presumptive positives was a confirmed positive, while one of the luminometrically-positive samples was a confirmed negative. These data demonstrate the utility of this assay as a near real-time screening tool for Listeria detection.

TABLE 1
Summary Table for Environmental Analysis

| Sample | Luminometer | IMS/PALCAM | BAM | S:B |
|---|---|---|---|---|
| TS-1 | Presumptive | — | — | 1.1 |
| TS-2 | — | — | — | 0.6 |
| TS-3 | — | — | — | 0.7 |
| TS-4 | — | — | — | 0.8 |
| TS-5 | — | — | — | 0.7 |
| TS-6 | — | — | — | 0.9 |
| TS-7 | + | — | — | 3.3 |
| TS-8 | Presumptive | — | — | 1.2 |
| TS-9 | + | — | — | 20.1 |
| TS-10 | + | — | — | 1.2 |

Luminometer = β-glucosidase assay of Example 2.
IMS/PALCAM = immuno-magnetic capture as described in Example 3, followed by direcv plating on PALCAM agar.
BAM = Bacteriological Assay Manual method of FDA, pre-enrichment in Listeria Enrichment broth, followed by enrichment in Fraser broth, plating positive samples on PALCAM agar.
S:B = signal to background ration.

### Example 4

### Effect of Aging the Substrate

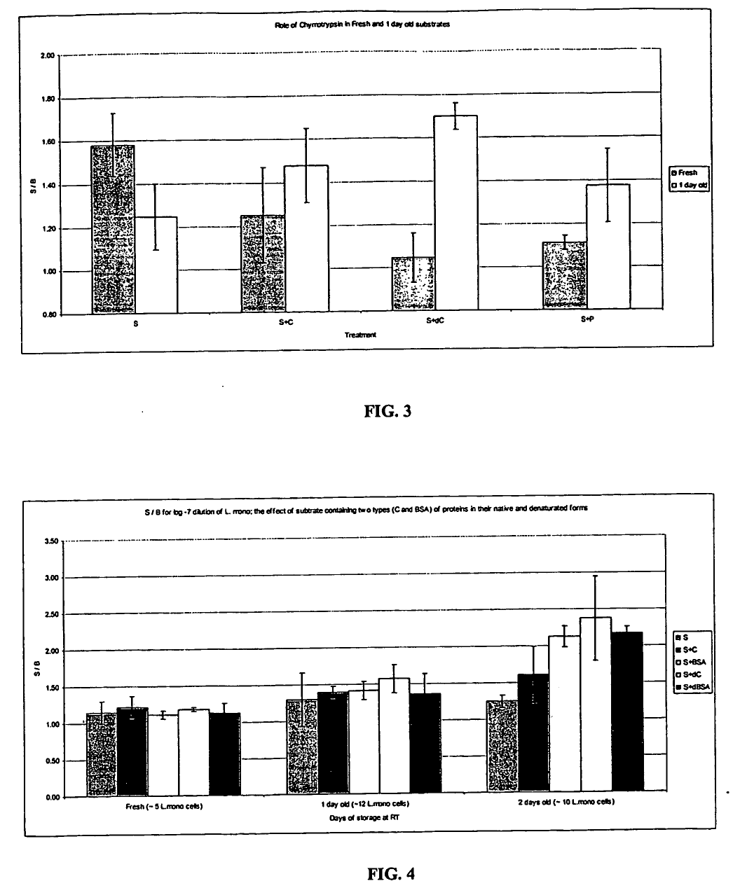

Interestingly, it has been found that the chemiluminescent substrates described herein exhibit greater sensitivity if the substrate is “aged” in the presence of proteins (generally) and in the presence off heat-denatured proteins (specifically). See FIGS. 3 and 4. The data in FIG. 3 and 4 demonstrate that Listeria monocytogenes cells that have been immuno-magnetically captured are more easily detectable in substrate preparations aged at room temperature in a solution with denatured proteins as compared to the same approach using freshly-prepared substrate solutions.

In FIG. 3 is presented signal to noise data for identical protocols that were run in duplicate, once using fresh solutions, and once using solutions aged for 24 hours.

The data presented in FIG. 3 were generated by a series of experiments that were designed to test the role of chymotrypsin in chemiluminescent detection of Listeria spp.

The treatments tested for the data presented in FIG. 3 were as follows:

1. (S): Substrate (0.8 ug/mL dioxetane-glucoside in pH 7.0 buffer)

2. (S+C): Substrate plus chymotrypsin ( 0.2 mg/mL chymotrypsin in substrate)

3. (S+dC): Substrate plus denaturated chymotrypsin (0.2 mg/mL denaturated chymotrypsin (held at 55° C. for thirty minutes) in substrate

4. (S+P): Substrate plus peptone (0.1 mg/mL peptone in substrate)

The data presented in FIG. 4 was generated using the same procedures as for the data in FIG. 3, with the exception that roughly ⅓ of the amounts of chymotrypsin and BSA were used.

The treatments tested for the data presented in FIG. 3 were as follows:

1. (S): Substrate (0.8 ug/mL dioxetane-glucoside in pH 7.0 buffer)

2. (S+C): Substrate plus chymotrypsin (0.06 mg/mL chymotrypsin in substrate)

3. (S+dC): Substrate plus denaturated chymotrypsin (0.06 mg/mL denaturated chymotrypsin (held at 55° C. for thirty minutes) in substrate

4. (S+BSA): Substrate plus bovine serum albumin (0.046 mg/mL BSA in substrate)

5. (S+dBSA): Substrate plus denaturated bovine serum albumin (0.046 mg/mL denaturated BSA (held at 55° C. for thirty minutes) in substrate

Notes:

The number of Listeria spp. measured was 5-12 CFU/mL.

Signal/Background (S/B) was calculated by dividing the RLU value of a sample to that of corresponding (−) control, peptone.

Analyses were done at least in duplicates.

The number of Listeria spp. was estimated based upon the plating of a log⁻⁷ dilution of an overnight L. monocytogenes culture in Tryptic Soy Broth onto PALCAM plates.

As shown in FIGS. 3 and 4, including denatured proteins in the substrate preparations increases the sensitivity at the limit of detection by a factor of about 2. Using this approach, the present invention can routinely detect as few as five (5) to ten (10) bacterial cells, in real time.

What is claimed is:

1. A method for detecting Listeria spp. in a sample, the method comprising:
(a) providing an inert surface having adhered thereto anti-Listeria antibodies capable of capturing Listeria spp. cells;
(b) contacting the surface of step (a) with a sample suspected of containing Listeria spp., wherein Listeria spp. cells present in the sample adhere to the anti-Listeria antibodies on the surface;
(c) contacting the surface of step (b) with a substrate for beta-glucosidase that produces luminescence when hydrolyzed, wherein beta-glucosidase produced by the Listeria spp. cells adhered to the anti-Listeria antibodies catalyzes hydrolysis of the substrate; and
(d) contacting the surface of step (c) with an enhancer molecule, and then
(e) detecting the luminescence generated in step (c), wherein the luminescence is indicative of the presence of the Listeria spp. cells in the sample.

2. The method of claim 1, wherein in step (a), the inert surface is a particle.

3. The method of claim 1, wherein in step (a), the inert surface is a magnetic particle.

4. The method of claim 1, wherein in step (a), the inert surface is a silica-coated particle.

5. The method of claim 1, wherein in step (a), the inert surface is a dextran-coated particle.

6. The method of claim 1, wherein in step (a), the inert surface is a silica-and dextran-coated particle.

7. The method of claim 1, wherein in step (a), the inert surface has adhered thereto anti-Listeria IgG.

8. The method of claim 1, wherein in step (c), the substrate for beta-glucosidase comprises a 1,2-dioxetane.

9. The method of claim 8, wherein in step (c), the substrate for beta-glucosidase comprises a compound selected from the group consisting of {(4-(2-phenoxyethoxy)-4-(3-phosphoryloxy-4-chlorophenyl)} spiro (1,2-dioxetane-3,13′-tricyclo(7.3.1.0^{2,7}}tridec-2,7-ene} and salts thereof

10. The method of claim 1, wherein in step (d), the enhancer molecule comprises a co-polymer of styrene and a polymerizable quaternary ammonium monomer.

11. The method of claim 1, wherein in step (d), the enhancer molecule comprises a poly(vinylbenzyl) ammonium polymer having an weight average molecular weight (M_{w}) of from about 50,000 to 70,000 Da.

12. The method of claim 1, wherein in step (d), the enhancer molecule is selected from the group consisting of compounds of Formula I and Formula II: Formula I: 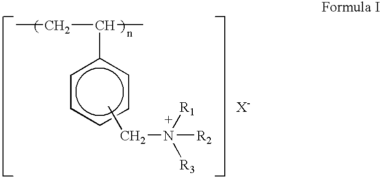
wherein each of R₁, R₂ and R₃ can be a straight or branched chain unsubstituted alkyl group having from 1 to 20 carbon atoms, a straight or branched chain alkyl group having from 1 to 20 carbon atoms substituted with one or more hydroxy, alkoxy, aryloxy, amino, substituted amino, amido, fluoroalkane, or fluoroaryl groups; an unsubstituted monocycloalkyl group having from 3 to 12 ring carbon atoms, a substituted monocycloalkyl group having from 3 to 12 ring carbon atoms substituted with one or more alkyl, alkoxy or fused benzo groups; a polycycloalkyl group having 2 or more fused rings, each having from S to 12 carbon atoms unsubstituted or substituted with one or more alkyl, alkoxy or aryl groups; an aryl, alkaryl or aralkyl group having at least one ring and from 6 to 20 carbon atoms in toto, unsubstituted or substituted with one or more alkyl, aryl, or fluoroalkane or fluoroaryl groups;
X— is a counterion; and
“n” is a positive integer such that the molecular weight of the Formula I compound will range from about 800 to about 200,000 Da; and
water-soluble acetals of a polyvinylalcohol and a formylbenzyl quaternary ammonium salt as shown in Formula II: 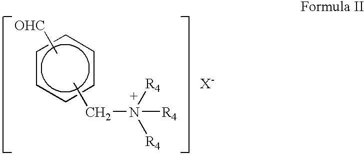
wherein each R₄ is the same or a different aliphatic substituent and X— is an anion.

13. The method according to any one of claims 1 to 12, further comprising, prior to step (c) aging the substrate at room temperature in the presence of proteins for a period of at least 12 hours.

14. The method according to claim 13, comprising aging the substrate at room temperature for a period of at least 24 hours.

15. The method according to claim 13, comprising aging the substrate at room temperature for a period of at least 48 hours.

16. The method according to claim 13, comprising aging the substrate in the presence of heat-denatured proteins.

17. The method according to any one of claims 1 to 12, further comprising, after step (b) and prior to step (c), separating the surface from the sample.

18. A kit for detecting Listeria spp. in a sample, the kit comprising:
an inert surface having adhered thereto anti-Listeria antibodies capable of capturing Listeria spp. cells;
a substrate for beta-glucosidase that produces luminescence when hydrolyzed, wherein the substrate is disposed in a first container;
an enhancer molecule disposed in a second container; and
instructions for use of the kit.

19. The kit of claim 18, wherein the inert surface is a particle.

20. The kit of claim 18, wherein the inert surface is a magnetic particle.

21. The kit of claim 18, wherein the inert surface is a silica-coated particle.

22. The kit of claim 18, wherein the inert surface is a dextran-coated particle.

23. The kit of claim 18, wherein the inert surface is a silica- and dextran-coated particle.

24. The kit of claim 18, wherein the inert surface has adhered thereto anti-Listeria IgG.

25. The kit of claim 18, wherein the substrate for beta-glucosidase comprises a 1,2-dioxetane.

26. The kit of claim 18, wherein the substrate for beta-glucosidase comprises a compound selected from the group consisting of {(4-(2-phenoxyethoxy)-4-(3-phosphoryloxy4-chlorophenyl)} spiro {1,2-dioxetane-3,13′-tricyclo{7.3.1.0^{2,7}}tridec-2,7-ene} and salts thereof.

27. The kit of claim 18, the enhancer molecule comprises a co-polymer of styrene and a polymerizable quaternary ammonium monomer.

28. The kit of claim 18, wherein the enhancer molecule comprises a poly(vinylbenzyl) ammonium polymer having an weight average molecular weight (M_{w}) of from about 50,000 to 70,000 Da.

29. The kit of claim 18, wherein the enhancer molecule is selected from the group consisting of compounds of Formula I: 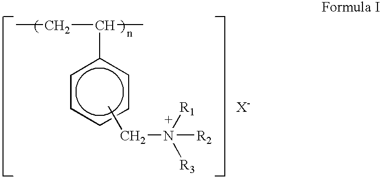
wherein each of R₁, R₂ and R₃ can be a straight or branched chain unsubstituted alkyl group having from 1 to 20 carbon atoms, a straight or branched chain alkyl group having from 1 to 20 carbon atoms substituted with one or more hydroxy, alkoxy, aryloxy, amino, substituted amino, amido, fluoroalkane, or fluoroaryl groups; an unsubstituted monocycloalkyl group having from 3 to 12 ring carbon atoms, a substituted monocycloalkyl group having from 3 to 12 ring carbon atoms substituted with one or more alkyl, alkoxy or fused benzo groups; a polycycloalkyl group having 2 or more fused rings, each having from 5 to 12 carbon atoms unsubstituted or substituted with one or more alkyl, alkoxy or aryl groups; an aryl, alkaryl or aralkyl group having at least one ring and from 6 to 20 carbon atoms in toto, unsubstituted or substituted with one or more alkyl, aryl, or fluoroalkane or fluoroaryl groups;
X— is a counterion; and
“n” is a positive integer such that the molecular weight of the Formula I compound will range ftom about 800 to about 200,000 Da; and
water-soluble acetals of a polyvinylalcohol and a formylbenzyl quaternary ammonium salt as shown in Formula II: 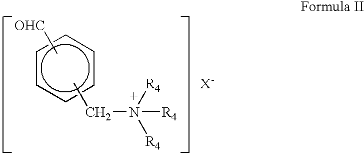
wherein each R∝is the same or a different aliphatic substituent and X— is an anion.
